SEQUENCE LISTING (5 sequences)

SEQ ID NO: 1 - DNA, 1192 bp, synthetic construct
  misc_feature 1..1192: note = Synthetic
  source 1..1192: mol_type = other DNA
cacctgaggtcaggagttcgagaccagcgtggccaacatgatgaaaccccgtctctagtaaaaatacaaaaattagccaggcatggtgctatatacctgtagcaccagctacttgggagacagaggtgggagaattacttgaacctgggaggttcaagccatgggaggtggaagttgcagtgagccgagatgccactgcactccagcctgagcaacagagcaagactatctcaagaaaagaaagaaagaaagaaagagacttgccaaggtcatgtatcagggcaaggaagagctgggggcccagctggctgctcccctgctgagctgggagaccaccttgatctgacttctcccatcttcccagcctaagccaggccctggggtcacggaggctggggaggcaccgaggaacgcgcctggcatgtgctgacaggggattttatgctccagctgggccagctgggaggagcctgctgggcagaggccagagctgggggctctggaaggtacctgggggaggttgcactgtgagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtg

SEQ ID NO: 2 - DNA, 589 bp, synthetic construct
  misc_feature 1..589: note = Synthetic
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 3 - DNA, 225 bp, synthetic construct
  misc_feature 1..225: note = Synthetic
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 4 - DNA, 5024 bp, synthetic construct
  misc_feature 1..5024: note = Synthetic
  source 1..5024: mol_type = other DNA
aggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtgatggccctggggacgacgctcagggcttctctcctgctcctggggctgctgactgaaggcctggcgcagttggcgattcctgcctccgttccccggggcttctgggccctgcctgaaaacctgacggtggtggagggggcctcagtggagctgcgttgtggggtcagcacccctggcagtgcggtgcaatgggccaaagatgggctgctcctgggccccgaccccaggatcccaggcttcccgaggtaccgcctggaaggggaccctgctagaggtgaattccacctgcacatcgaggcctgtgacctcagcgatgacgcggagtatgagtgccaggtcggccgctctgagatggggcccgagctcgtgtctcccagagtgatcctctccatcctggttcctcccaagctgctcctgctgaccccagaggcaggcaccatggtcacctgggtagctgggcaggagtacgtggtcaactgtgtgtctggggacgcgaagccagcacctgacatcaccattctcctgagtggacagacaatatctgacatctctgcaaacgtgaacgagggctcccagcagaaactcttcactgtggaggccacagccagggtgacaccccggagctcagataataggcagttgctggtctgtgaggcgtctagcccagcactggaggcccccatcaaggcctcattcaccgtgaatgttctgttccctccaggaccccctgtcatcgagtggccaggcctggatgaggggcacgtgcgggcaggacagagcttggagctgccgtgcgtggcccgagggggtaatcccttagccacactgcagtggctgaagaatggccagccggtgtccacagcgtggggcacagagcacacccaggcggtggcccgcagtgtgctggtgatgaccgtgaggccagaagaccatggagcgcagctcagctgcgaggcccacaacagcgtgtctgcagggacccaggagcacggcatcacactgcaggtcacctttccccctagtgccattattatcttgggatctgcatcccagactgagaacaagaacgtgacactctcctgtgtcagcaagtccagtcgcccgcgggttctgctacgatggtggctgggctggcggcagctgctgcccatggaggagacagtcatggatggactgcatggcggtcacatctccatgtccaacctgacattcctggcgcggcgggaggacaacggtctgaccctcacatgtgaggccttcagtgaagccttcaccaaggagaccttcaagaagtcgctcatcctgaacgtaaaatatcccgcccagaaactgtggattgagggtcccccagagggccagaagctccgggctgggacccgggtgaggctggtgtgtttggctatcgggggcaacccagagccctccctcatgtggtacaaggactcgcgcaccgtgaccgagtcgcggctgccgcaggagtcgcggcgcgtgcatctcggcagcgtggagaaatctgggagcaccttctcccgagagctggtgctggtcacagggccgtcggacaaccaggccaagttcacgtgcaaggctggacagctcagcgcgtccacgcagctggcggtgcagtttcccccaactaacgtgacgatcctggccaacgcatccgcactgcgcccgggagacgccttaaacttgacatgcgtcagcgtcagcagcaatccgccggtcaacttgtcctgggacaaggaaggggagaggctggagggcgtggccgccccaccccggagagccccattcaaaggctccgccgccgccaggagcgtccttctgcaagtgtcatcccgcgatcatggccagcgcgtgacctgccgcgcccacagcgccgagctccgcgaaaccgtgagctccttctatcgcctcaacgtactgtaccgtccagagttcctgggggagcaggtgctggtggtgaccgcggtggagcagggcgaggcgttgctgcccgtgtccgtgtccgctaaccccgcccccgaggccttcaactggaccttccgcggctatcgcctcagtccagcgggcggcccccggcatcgcatcctgtccagcggggctctgcatctgtggaatgtgacccgcgcggacgacggcctctatcagctgcactgccagaactctgagggcaccgcggaagcgcggctgcggctggacgtgcactatgctcccaccatccgtgccctccaggaccccactgaggtgaacgtcgggggttctgtggacatagtctgcactgtcgatgccaatcccatcctcccgggcatgttcaactgggagagactgggagaagatgaggaggaccagagcctggatgacatggagaagatatccaggggaccaacggggcgcctgcggattcaccatgccaaactggcccaggctggcgcttaccagtgcattgtggacaatggggtggcgcctccagcacgacggctgctccgtcttgttgtcagatttgccccccaggtggagcaccccactcccctaactaaggtggctgcagctggagacagcaccagttctgccaccctccactgccgtgcccgaggtgtccccaacatcgttttcacttggacaaaaaacggggtccctctggatctccaagatcccaggtacacggagcacacataccaccagggtggtgtccacagcagcctcctgaccattgccaacgtgtctgccgcccaggattacgccctcttcacatgtacagccaccaacgcccttggctcggaccaaaccaacattcaacttgtcagcatcagccgccctgaccctccatcaggattaaaggttgtgagtctgaccccacactccgtggggctggagtggaagcctggctttgatgggggcctgccacagaggttctgcatcaggtatgaggccctggggactccagggttccactatgtggatgtcgtaccaccccaggccaccaccttcacgctgactggtctacagccttctacaagatacagggtctggctgctggccagtaatgccttgggggacagtggactggctgacaaagggacccagcttcccatcactaccccaggtctccaccagccttctggagaacctgaagaccagctgcccacagagccaccttcaggaccctcggggctgcccctgctgcctgtgctgttcgctcttggggggcttctgctcctctccaatgcctcctgtgtcgggggggtcctctggcagcggagactcaggcgtcttgctgagggcatctcagagaagacagaggcagggtcggaagaggaccgagtcaggaacgaatatgaggagagccagtggacaggagagcgggacactcagagctccacggtcagcacaacagaggcagagccgtattaccgctccctgagggacttcagcccccagctgcccccgacgcaggaggaggtgtcttattcccgaggtttcacaggtgaagatgaggatatggccttccctgggcacttgtatgatgaggtagaaagaacgtaccccccgtctggagcctggggacccctctacgatgaagtgcagatgggaccctgggacctccactggcctgaagacacatatcaggatccaagaggaatctatgaccaggtggccggagacttggacactctggaacccgattctctgcccttcgagctgaggggacatctggtgtaagagccctctcaaccccattgtcctgcacctgcaggaatttacactccactggtctctctcattacagcctgggccgagctggttaggtgagctccataaaacccaaagggacttggtgtcaggagaggacatggagggggctgagtgacagagatggttcagctggtaccagagtagaaacaaggtgcatcctggggttggctttagaaactaaacttctccaaaaggacagggcagattgtaaacgtcgtctcaaaaatgaaatgctgccgggtgcggtgactcacgcctataatcccagcactttgggaggctgaggcgggtggatcacctgaggtcaggagttcgagaccagcctggccaacatggtaaaactccatttctactaaaaatataaaaaattagccaggagtagtggcgcatgcctgtagtcccagctacttgggaggctgatgcatgagaattgcttgaacccaggaggcggaggttgcagtgagctgagatcacgccactgcactccagcctgggcgacagagcgagattctgtctcaaaaaataaaaataaaataaaaataaaaaataaaatgctattctgagggtccctaagtgcttcatgaagtaaagttttcagaagtggggaaagacatatgaaaaactctgttcattttgcattccatgacgtctgaaatagctaataataagtggaatttatgaatccatgttgcatcctacctattattttattatttccgacagttatctaacacaggaggcataaactatgacacatgtaaacataagtatgctccataagcattaaaataaacagacaatcaaaagaagctagaggaaaagaatatgtactgtgtgtgtgctttcgtttatagaaattctaaaataggcaaaactaatctacggtggcagagagcacagcagaggtttcctggggccatgagaggagggttgactgcaaaaaggcatgggagaactttctgaggtgttggaaatattctatataacgattgtggtggtggttgcacgggtgcacagatttgtcaaaactcattgaactttacacttaagatgggtgcatttttattgtacataaatgatatctcaataaaagtgattttaaaaacagg

SEQ ID NO: 5 - DNA, 5535 bp, synthetic construct
  misc_feature 1..5535: note = Synthetic
  source 1..5535: mol_type = other DNA
gagaatgagctcaagctgggtcagagagcagggctgactctgccagtgcctgcatcagcctcatcgctctcctaggctcctggcctgctggactctgggctgcaggtccttcttgaaaggctgtgagtagtgagacaaggagcaggagtgaggggtggcaggagagaagatagagattgagagagagagagagagagagacagagagagaggaagagacagagacaaaaggagagagaacggcttagacaaggagagaaagatggaaagataaagagactgggcgcagtggctcacgcctgtaatcccaacacttggggaggccaaggtgggaggatggcttgaaggaaagagtctgagatcaacctggccaacatagtgagaccccgtctctaaaaaaaaaaaaaagaaaaaaaaaagaaaaaagaaaaaaaagtttttttaaagagacagagaaagagactcagagattgagactgagagcaagacagagagagatactcacagggaagaggggaagaggaaaacgagaaagggaggagagtaacggaaagagataaaaaagaaaagcaggtggcagagacacacagagagggacccagagaaagccagacagacgcaggtggctggcagcgggcgctgtgggggtcacagtagggggacctgtgatggccctggggacgacgctcagggcttctctcctgctcctggggctgctgactgaaggcctggcgcagttggcgattcctgcctccgttccccggggcttctgggccctgcctgaaaacctgacggtggtggagggggcctcagtggagctgcgttgtggggtcagcacccctggcagtgcggtgcaatgggccaaagatgggctgctcctgggccccgaccccaggatcccaggcttcccgaggtaccgcctggaaggggaccctgctagaggtgaattccacctgcacatcgaggcctgtgacctcagcgatgacgcggagtatgagtgccaggtcggccgctctgagatggggcccgagctcgtgtctcccagagtgatcctctccatcctggttcctcccaagctgctcctgctgaccccagaggcaggcaccatggtcacctgggtagctgggcaggagtacgtggtcaactgtgtgtctggggacgcgaagccagcacctgacatcaccattctcctgagtggacagacaatatctgacatctctgcaaacgtgaacgagggctcccagcagaaactcttcactgtggaggccacagccagggtgacaccccggagctcagataataggcagttgctggtctgtgaggcgtctagcccagcactggaggcccccatcaaggcctcattcaccgtgaatgttctgttccctccaggaccccctgtcatcgagtggccaggcctggatgaggggcacgtgcgggcaggacagagcttggagctgccgtgcgtggcccgagggggtaatcccttagccacactgcagtggctgaagaatggccagccggtgtccacagcgtggggcacagagcacacccaggcggtggcccgcagtgtgctggtgatgaccgtgaggccagaagaccatggagcgcagctcagctgcgaggcccacaacagcgtgtctgcagggacccaggagcacggcatcacactgcaggtcacctttccccctagtgccattattatcttgggatctgcatcccagactgagaacaagaacgtgacactctcctgtgtcagcaagtccagtcgcccgcgggttctgctacgatggtggctgggctggcggcagctgctgcccatggaggagacagtcatggatggactgcatggcggtcacatctccatgtccaacctgacattcctggcgcggcgggaggacaacggtctgaccctcacatgtgaggccttcagtgaagccttcaccaaggagaccttcaagaagtcgctcatcctgaacgtaaaatatcccgcccagaaactgtggattgagggtcccccagagggccagaagctccgggctgggacccgggtgaggctggtgtgtttggctatcgggggcaacccagagccctccctcatgtggtacaaggactcgcgcaccgtgaccgagtcgcggctgccgcaggagtcgcggcgcgtgcatctcggcagcgtggagaaatctgggagcaccttctcccgagagctggtgctggtcacagggccgtcggacaaccaggccaagttcacgtgcaaggctggacagctcagcgcgtccacgcagctggcggtgcagtttcccccaactaacgtgacgatcctggccaacgcatccgcactgcgcccgggagacgccttaaacttgacatgcgtcagcgtcagcagcaatccgccggtcaacttgtcctgggacaaggaaggggagaggctggagggcgtggccgccccaccccggagagccccattcaaaggctccgccgccgccaggagcgtccttctgcaagtgtcatcccgcgatcatggccagcgcgtgacctgccgcgcccacagcgccgagctccgcgaaaccgtgagctccttctatcgcctcaacgtactgtaccgtccagagttcctgggggagcaggtgctggtggtgaccgcggtggagcagggcgaggcgttgctgcccgtgtccgtgtccgctaaccccgcccccgaggccttcaactggaccttccgcggctatcgcctcagtccagcgggcggcccccggcatcgcatcctgtccagcggggctctgcatctgtggaatgtgacccgcgcggacgacggcctctatcagctgcactgccagaactctgagggcaccgcggaagcgcggctgcggctggacgtgcactatgctcccaccatccgtgccctccaggaccccactgaggtgaacgtcgggggttctgtggacatagtctgcactgtcgatgccaatcccatcctcccgggcatgttcaactgggagagactgggagaagatgaggaggaccagagcctggatgacatggagaagatatccaggggaccaacggggcgcctgcggattcaccatgccaaactggcccaggctggcgcttaccagtgcattgtggacaatggggtggcgcctccagcacgacggctgctccgtcttgttgtcagatttgccccccaggtggagcaccccactcccctaactaaggtggctgcagctggagacagcaccagttctgccaccctccactgccgtgcccgaggtgtccccaacatcgttttcacttggacaaaaaacggggtccctctggatctccaagatcccaggtacacggagcacacataccaccagggtggtgtccacagcagcctcctgaccattgccaacgtgtctgccgcccaggattacgccctcttcacatgtacagccaccaacgcccttggctcggaccaaaccaacattcaacttgtcagcatcagccgccctgaccctccatcaggattaaaggttgtgagtctgaccccacactccgtggggctggagtggaagcctggctttgatgggggcctgccacagaggttctgcatcaggtatgaggccctggggactccagggttccactatgtggatgtcgtaccaccccaggccaccaccttcacgctgactggtctacagccttctacaagatacagggtctggctgctggccagtaatgccttgggggacagtggactggctgacaaagggacccagcttcccatcactaccccaggtctccaccagccttctggagaacctgaagaccagctgcccacagagccaccttcaggaccctcggggctgcccctgctgcctgtgctgttcgctcttggggggcttctgctcctctccaatgcctcctgtgtcgggggggtcctctggcagcggagactcaggcgtcttgctgagggcatctcagagaagacagaggcagggtcggaagaggaccgagtcaggaacgaatatgaggagagccagtggacaggagagcgggacactcagagctccacggtcagcacaacagaggcagagccgtattaccgctccctgagggacttcagcccccagctgcccccgacgcaggaggaggtgtcttattcccgaggtttcacaggtgaagatgaggatatggccttccctgggcacttgtatgatgaggtagaaagaacgtaccccccgtctggagcctggggacccctctacgatgaagtgcagatgggaccctgggacctccactggcctgaagacacatatcaggatccaagaggaatctatgaccaggtggccggagacttggacactctggaacccgattctctgcccttcgagctgaggggacatctggtgtaagagccctctcaaccccattgtcctgcacctgcaggaatttacactccactggtctctctcattacagcctgggccgagctggttaggtgagctccataaaacccaaagggacttggtgtcaggagaggacatggagggggctgagtgacagagatggttcagctggtaccagagtagaaacaaggtgcatcctggggttggctttagaaactaaacttctccaaaaggacagggcagattgtaaacgtcgtctcaaaaatgaaatgctgccgggtgcggtgactcacgcctataatcccagcactttgggaggctgaggcgggtggatcacctgaggtcaggagttcgagaccagcctggccaacatggtaaaactccatttctactaaaaatataaaaaattagccaggagtagtggcgcatgcctgtagtcccagctacttgggaggctgatgcatgagaattgcttgaacccaggaggcggaggttgcagtgagctgagatcacgccactgcactccagcctgggcgacagagcgagattctgtctcaaaaaataaaaataaaataaaaataaaaaataaaatgctattctgagggtccctaagtgcttcatgaagtaaagttttcagaagtggggaaagacatatgaaaaactctgttcattttgcattccatgacgtctgaaatagctaataataagtggaatttatgaatccatgttgcatcctacctattattttattatttccgacagttatctaacacaggaggcataaactatgacacatgtaaacataagtatgctccataagcattaaaataaacagacaatcaaaagaagctagaggaaaagaatatgtactgtgtgtgtgctttcgtttatagaaattctaaaataggcaaaactaatctacggtggcagagagcacagcagaggtttcctggggccatgagaggagggttgactgcaaaaaggcatgggagaactttctgaggtgttggaaatattctatataacgattgtggtggtggttgcacgggtgcacagatttgtcaaaactcattgaactttacacttaagatgggtgcatttttattgtacataaatgatatctcaataaaagtgattttaaaaacagg

(19) United States
(12) Patent Application Publication
(10) Pub. No.: US 2025/0064975 A1
(43) Pub. Date: Feb. 27, 2025

(54) THERAPY

(71) Applicant: The University of Bristol, Bristol (GB)

(72) Inventors: Moin Ahson Saleem-Uddin, Bristol (GB); Gavin Iain Welsh, Bristol (GB); Wen Yi Ding, Swindon (GB)

(21) Appl. No.: 18/886,555

(22) Filed: Sep. 16, 2024

Related U.S. Application Data
(63) Continuation of application No. 17/348,048, filed on Jun. 15, 2021, now Pat. No. 12,121,593.

(30) Foreign Application Priority Data
Jun. 15, 2020 (GB) 2009039.5

(51) Int. Cl. A61K 48/00; C12N 15/86

(52) U.S. Cl. CPC A61K 48/005; C12N 15/86; C12N 2750/14143; C12N 2830/48; C12N 2830/50

(57) ABSTRACT

The application provides gene therapies for treating monogenic forms of nephrotic syndrome.

### CROSS-REFERENCE TO RELATED APPLICATIONS

This application is a Continuation of U.S. application Ser. No. 17/348,048, filed Jun. 15, 2021, which claims priority to United Kingdom Application No. 2009039.5, filed Jun. 15, 2020, both of which are incorporated herein by reference in their entirety.

### INCORPORATION BY REFERENCE OF THE SEQUENCE LISTING

This application contains, as a separate part of disclosure, a Sequence Listing in computer-readable form (Filename: 56901A_SeqListing.xml; 19,727 bytes—XML file created Sep. 16, 2024) which is incorporated by reference herein in its entirety.

### FIELD OF INVENTION

The present invention relates to gene therapies for use in treating monogenic forms of nephrotic syndrome.

### BACKGROUND TO THE INVENTION

Nephrotic syndrome (NS) is a chronic kidney disease characterized by significant proteinuria, hypoalbuminemia, oedema and hyperlipidemia, and is the most common primary glomerular disease in children, affecting 2/100,000 children below the age of 16 in Europe and the USA. NS is associated with different ages of onset, from less than 3 months old at diagnosis to early adulthood, and is classified in different patients groups depending on their sensitivity to corticoids: ˜80% of children with NS are classified as having steroid-sensitive nephrotic syndrome (SSNS) and can be successfully treated with corticosteroid therapy. A proportion of patients originally classified as SSNS relapse and need further steroid treatment, and a further 10-15% of NS patients do not achieve remission after weeks of therapy with corticosteroids and are classified as having steroid-resistant nephrotic syndrome (SRNS). Up to 50% of these SRNS patients progress to end-stage renal disease within 10 years and commonly face an elevated risk of recurrence after renal transplant, highlighting the lack of suitable and efficient treatment for these patients.

Podocyte dysfunction and resultant disruption of the glomerular filtration barrier is central to the pathogenesis of NS. The podocyte branches off cellular processes to cover the outside of the glomerular capillary, called foot processes, and their interdigitations with neighbouring foot processes form the glomerular slit membrane, critical for the glomerular filtration barrier efficiency and for the retention of protein in the blood stream. In genetic forms of NS, mutations in genes coding for key podocyte processes such as the development, migration, basement membrane interaction, or regeneration of the podocyte, lead to the loss of integrity of the glomerular slit membrane and to the nephrotic syndrome phenotype. Approximately 30% of cases of SRNS in children are genetic, where the most common mutations in childhood are in NPHS2 encoding podocin, accounting for 10-30% of sporadic genetic cases.

Podocin is a 42 kDa hairpin like membrane-associated podocyte-specific protein that is a key component of the protein complex at the slit diaphragm; the cell-cell junction between adjacent podocyte foot processes. It localises to lipid rafts and interacts with other important slit diaphragm proteins like nephrin, CD2AP and TRPC6. It is essential in the maintenance of the slit diaphragm, and consequently the integrity of the glomerular filtration barrier. There are 126 mutations reported to date, but the most common mutation is R138Q, which causes mislocalisation of podocin to the endoplasmic reticulum.

As no effective treatment currently exists for patients with monogenic forms of NS, the use of gene therapy for the transfer of a functional gene copy in diseased podocytes could constitute a promising novel strategy to address monogenic forms of NS, reverse NS phenotype and correct kidney dysfunction. Indeed, US2003/0152954 generally suggested the use of viral vectors to deliver nucleic acids encoding polypeptides with podocin activity but failed to disclose or test any specific gene therapy constructs. It is likely that this is because the kidney has a complex anatomy with specialised compartments composed of glomeruli, tubules, vasculature, and interstitium, which makes it a difficult target for gene therapy vectors. To date renal-targeted gene therapy has been largely unsuccessful as the highly differentiated sub-structures of the kidney can be difficult to target and specifically transduce with viral vector approaches (van der Wouden et al., 2004).

A recent study attempted to target the kidney using rAAV vectors in combination with a CMV promoter and GFP or luciferase genes, which were administered via tail vein injection or renal vein injection (Rocca et al 2014). However, tail vein injection was shown to be unsuitable for kidney transduction and, while a low level of gene expression in podocytes was observed, widespread expression was also observed in the liver, despite the use of an allegedly kidney specific promoter. The study additionally failed to demonstrate successful transduction of a NS-associated transgene, such as podocin, and failed to demonstrate long term functional expression of such genes. The study also did not explore AAV serotypes suitable for human renal cell transduction.

The present invention aims to reverse the NS phenotype and correct podocyte-associated kidney dysfunction in patients with monogenic forms of NS by administering AAV gene therapy expressing a NS-associated transgene under the control of a podocyte-specific promoter.

### SUMMARY OF THE INVENTION

The present invention provides an adeno-associated virus (AAV) vector gene therapy comprising a NS-associated transgene and minimal nephrin promoter NPHS1 or podocin promoter NPHS2, wherein the NS-associated transgene is selected from one or more of NPHS1, TRPC6, NUP107, NUP133, NUP160, ACTN4, INF2, ANKFY1, ANLN, CRB2, ITGA3, KANK1, KANK4, MAGI2, MYO1E, OCRL, PTPRO, SMARCAL1, SYNPO, TBC1D8B, XPO5, TNS2 or NLRP3. The gene therapy vector can be used to target podocytes within the glomerulus of the kidney in order to reverse the NS phenotype and correct podocyte-associated kidney dysfunction, particularly in patients with monogenic forms of NS.

Suitable AAV vector serotypes include 2/9, LK03 and 3B.

The AAV 2/9 serotype has shown significant tropism for newborn and adult mouse kidney, localising to the glomeruli and tubules (Luo et al., 2011; Picconi et al., 2014; Schievenbusch et al., 2010), and AAV2/9 vector combined with renal vein injection has been shown to be suitable for kidney-targeted gene delivery (Rocca et al., 2014). AAV 2/9 is therefore one suitable vector for use in the gene therapy of the present invention.

Synthetic AAV capsids such as LK03 can also be suitable vectors for use in the gene therapy of the present invention. This vector has been shown to transduce human primary hepatocytes at high efficiency in vitro and in vivo. However, until now it has not been utilised in kidney-targeted gene delivery. Surprisingly, AAV-LK03 vectors can achieve high transduction of close to 100% in human podocytes in vitro and can be used to transduce podocytes specifically in vitro (see PCT/GB2020/050097, incorporated herein by reference).

The AAV-LK03 cap sequence consists of fragments from seven different wild-type serotypes (AAV1, 2, 3B, 4, 6, 8, 9), although AAV-3B represents 97.7% of the cap gene sequence and 98.9% of the amino acid sequence. AAV-3B is also known for its human hepatocyte tropism is another a suitable vector for use in the gene therapy of the present invention. To date it has not been utilised in kidney-targeted gene delivery.

The transgene species is preferably matched to the patient species. For example, when treating a human patient one would typically use a human transgene. The transgene may be naturally occurring, e.g. wild-type, or it may be recombinant. The transgene is typically included in the gene therapy vector as a cDNA sequence.

Use of a minimal nephrin promoter such as NPHS1 or podocin promoter NPHS2 allows the gene therapy vector to be targeted specifically to podocytes (Moeller et al., 2002; Picconi et al., 2014). This enables transgene expression to be specifically targeted to podocytes in the glomerular basement membrane of the kidney and minimises off-target expression. As podocytes are terminally differentiated and non-dividing cells they can be targeted for stable expression of the transgene and reduce or avoid any risk of vector dilution effect. In preferred embodiments of the invention the promoter is NPHS1. One example of a suitable DNA sequence for the NPHS1 promoter is shown in FIG. 1. The NPHS1 promoter may comprise a nucleic acid sequence which has at least 85%, 90%, 95%, 96%, 97%, 98%, 99% or more sequence identity to the sequence of FIG. 1, as long as it encodes a nephrin promoter that has retained its biological activity, particularly podocyte specific expression of a gene of interest or a reporter gene.

As with the transgene, the species of the promoter is preferably matched to the patient species. For example, when treating a human patient one would typically use human NHPS1 or human NPHS2.

The AAV vector may additionally comprise a Woodchuck hepatitis post-transcriptional regulatory element (WPRE). WPRE is a DNA sequence that, when transcribed, creates a tertiary structure enhancing expression. Inclusion of WPRE may increase expression of the transgene delivered by the vector. The WPRE sequence may be mutated to reduce oncogenicity without significant loss of RNA enhancement activity (Schambach et al., 2005, incorporated herein by reference). One example of a suitable WPRE sequence is shown in FIG. 2.

The NS-associated transgene may comprise a hemagglutinin (HA) tag. HA can be used as an epitope tag and has been shown not to interfere with bioactivity or biodistribution of proteins to which it has been added. The HA tag can facilitate detection, isolation, and purification of the transgene. Other suitable protein tags may include Myc tags, polyhistidine tags and flag tags.

The AAV vector may additionally comprise a Kozak sequence between the promoter and the podocin transgene. The Kozak sequence is known to play a major role in the initiation of the translation process and can therefore enhance expression of the NS-associated transgene.

The AAV vector may additionally comprise a polyadenylation signal, such as bovine growth hormone (bGH) polyadenylation signal, e.g., as shown in FIG. 3. Polyadenylation is the addition of a poly(A) tail to a messenger RNA. The poly(A) tail consists of multiple adenosine monophosphates; in other words, it is a stretch of RNA that has only adenine bases. The poly(A) tail is important for the nuclear export, translation, and stability of mRNA. Inclusion of a polyadenylation signal can therefore enhance expression of the NS-associated transgene.

The AAV vector gene therapy additionally typically comprises Inverted Terminal Repeat (ITR) sequences at either end of the vector. For example, the vector structure may be, in order: ITR-promotor-transgene (with optional HA tag)-optional WRPE-polyadenylation signal-ITR.

The gene therapy vector of the present invention can therefore be used to treat or manage monogenic forms of NS in a patient. The term “patient” as used herein may include any mammal, including a human. The patient may be an adult or a paediatric patient, such as a neonate or an infant. In embodiments of the invention the patient may be a paediatric patient between the ages of about 1 and about 16 years old.

The patient is suffering from a monogenic form of NS. In other words, the NS is caused by a mutation in one gene. Preferably mutation is in a gene expressed in podocytes, such as one or more mutations in any one of NPHS1, TRPC6, ACTN4, TRPC6, ANKFY1, ANLN, CRB2, ITGA3, KANK1, KANK4, MAGI2, MYO1E, NUP107, NUP133, NUP160, OCRL, PTPRO, SMARCAL1, SYNPO, TBC1D8B, XPO5, TNS2 or NLRP3. In embodiments of the invention the monogenic form of NS may be a monogenic form of SRNS caused by one or more mutations in any one of NPHS1, TRPC6, ACTN4 or TRPC6. For example, the NS may be SRNS caused by a mutation in NPHS1, which codes for nephrin, a type-1 transmembrane protein found at the slit diaphragm of glomerular podocytes. The slit diaphragm functions as an ultrafilter to exclude albumin and other plasma macromolecules in the formation of urine. Mutations in NPHS1 gene result in nephrotic syndrome, with the most common mutations being associated with Finnish-type congenital nephrosis, which is characterized by severe proteinuria and loss of the slit diaphragm and foot processes. The NPHS1 mutation may be a Fin-major mutation or a Fin-minor mutation (Kestilä et al).

The NS-associated transgene used in the gene therapy is a gene associated with a monogenic form of NS and expressed in podocytes, and which encodes a protein of about 1600 amino acids or less, optionally about 1500 amino acids or less, or about 1450 amino acids or less. This size limitation allows the NS-associated transgene to fit into the gene therapy vector of the present invention.

The transgene is typically included in the gene therapy vector as a cDNA sequence. However, the NS-associated transgene may be any polynucleotide, such as single or double-stranded DNA or RNA, comprising a nucleic acid sequence encoding any NS-associated polynucleotide as discussed above. For instance the NS-associated polynucleotide may comprise the NPHS1 open reading frame (ORF) sequence of FIG. 4A or 4B. The NPHS1 polynucleotide may comprise a nucleic acid sequence which has at least 85%, 90%, 95%, 96%, 97%, 98%, 99% or more sequence identity to the NPHS1 ORF sequence of FIG. 4A or 4B, as long as it encodes a nephrin polypeptide that has retained its biological activity, particularly its ability to maintain slit diaphragm integrity and/or slit diaphragm-mediated signalling. Nephrin protein consists of a C-terminal cytoplasmic domain, a transmembrane domain, a fibronectin type III-like module, eight extracellular Ig-like domains and a signal peptide domain. Preferably some or all of these structural components are conserved.

In the description above, the term “identity” is used to refer to the similarity of two sequences. For the purpose of this invention, it is defined here that in order to determine the percent identity of two sequences, the sequences are aligned for optimal comparison purposes (e.g., gaps can be introduced in the sequence of a first sequence for optimal alignment with a second amino or nucleic acid sequence). The nucleotide/amino acid residues at each position are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e. overlapping positions)×100). Generally, the two sequences are the same length. A sequence comparison is typically carried out over the entire length of the two sequences being compared.

The skilled person will be aware of the fact that several different computer programs are available to determine the identity between two sequences. For instance, a comparison of sequences and determination of percent identity between two sequences can be accomplished using a mathematical algorithm. For example, the percent identity between two nucleic acid sequences can be determined using the sequence alignment software Clone Manager 9 (Sci-Ed software—www.scied.com) using global DNA alignment; parameters: both strands; scoring matrix: linear (mismatch 2, OpenGap 4, ExtGap 1).

Alternatively, the percent identity between two amino acid or nucleic acid sequences can be determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6. A further method to assess the percent identity between two amino acid or nucleic acid sequences can be to use the BLAST sequence comparison tool available on the National Center for Biotechnology Information (NCBI) website (www.blast.ncbi.nlm.nih.gov), for example using BLASTn for nucleotide sequences or BLASTp for amino acid sequences using the default parameters.

The presence or absence of a monogenic form of NS can be determined by laboratory testing, such as that available from Bristol Genetics Laboratories, Bristol, UK. Typically, genetic testing can be performed by analysis of a blood sample obtained from the patient.

The AAV vector gene therapy may be administered systemically, such as by intravenous injection. In embodiments of the invention the AAV vector gene therapy may be administered by injection into the renal artery. In alternative embodiments of the invention the AAV vector gene therapy may be administered by retrograde administration, e.g. via the ureters using a urinary catheter.

The gene therapy may be administered as a single dose, in other words, subsequent doses of the vector may not be needed. In the event that repeated doses are needed different AAV serotypes can be used in the vector. For example, vector used in a first dose may comprise AAV-LK03 or AAV-3B whereas the vector used in a subsequent dose may comprise AAV 2/9.

Optionally the gene therapy may be administered in combination with temporary immunosuppression of the patient, e.g. by administering the gene therapy at the same time as, or following treatment with, oral steroids. Immunosuppression may be desirable before and/or during gene therapy treatment to suppress the patient's immune response to the vector. However, the AAV capsid is present only transiently in the transduced cell as it is not encoded by the vector. The capsid is therefore gradually degraded and cleared, meaning that a short-term immunomodulatory regimen that blocks the immune response to the capsid until capsid sequences are cleared from the transduced cells can allow long-term expression of the transgene. Immunosuppression may therefore be desirable for a period of about six weeks following administration of the gene therapy.

The AAV vector gene therapy may be administered in the form of a pharmaceutical composition. In other words the AAV vector gene therapy may be combined with one or more pharmaceutically acceptable carriers or excipients. A suitable pharmaceutical composition is preferably sterile.

### BRIEF DESCRIPTION OF THE DRAWINGS

The invention will now be described in detail, by way of example only, with reference to the figures.

FIG. 1 shows an example DNA sequence for the minimal human nephrin promoter (NPHS1).

FIG. 2 shows an example DNA sequence for a WPRE sequence.

FIG. 3 shows an example DNA sequence for a bGH poly(A) signal sequence.

FIGS. 4A and B show example cDNA sequences for human NPHS1 transgenes.

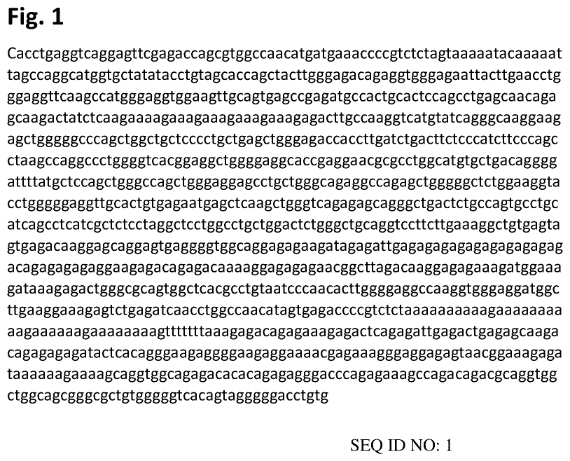

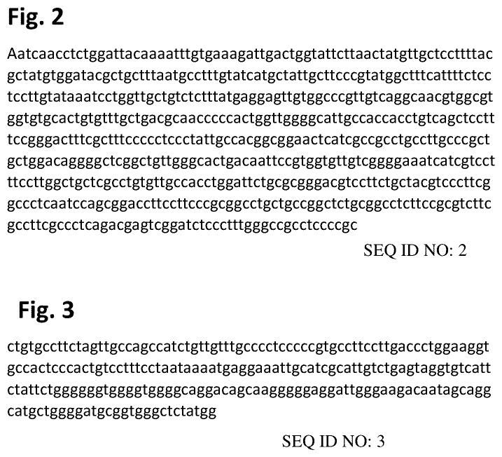

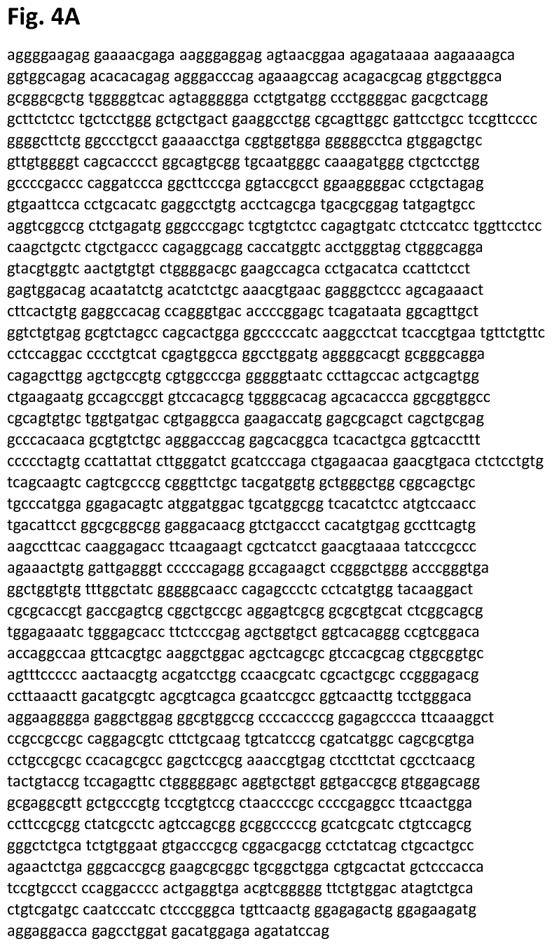

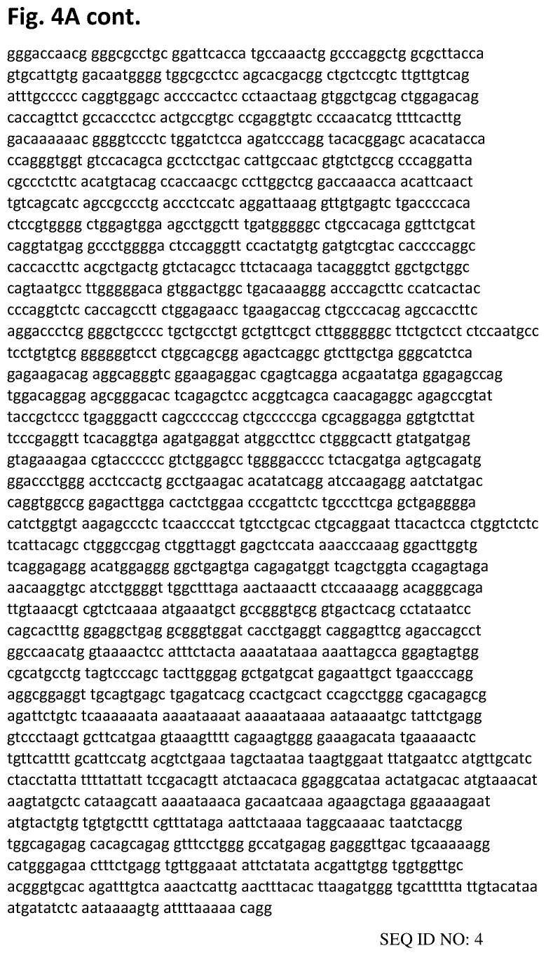

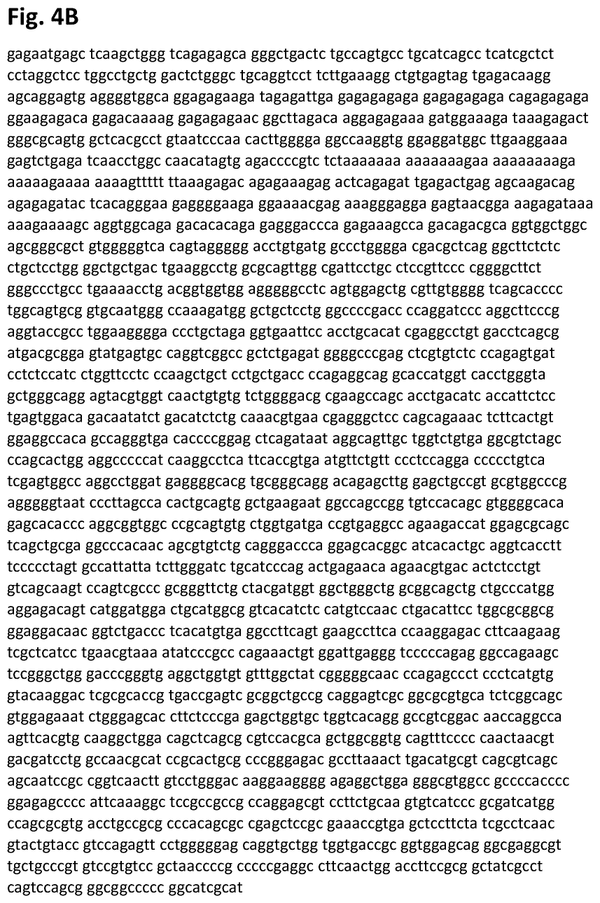

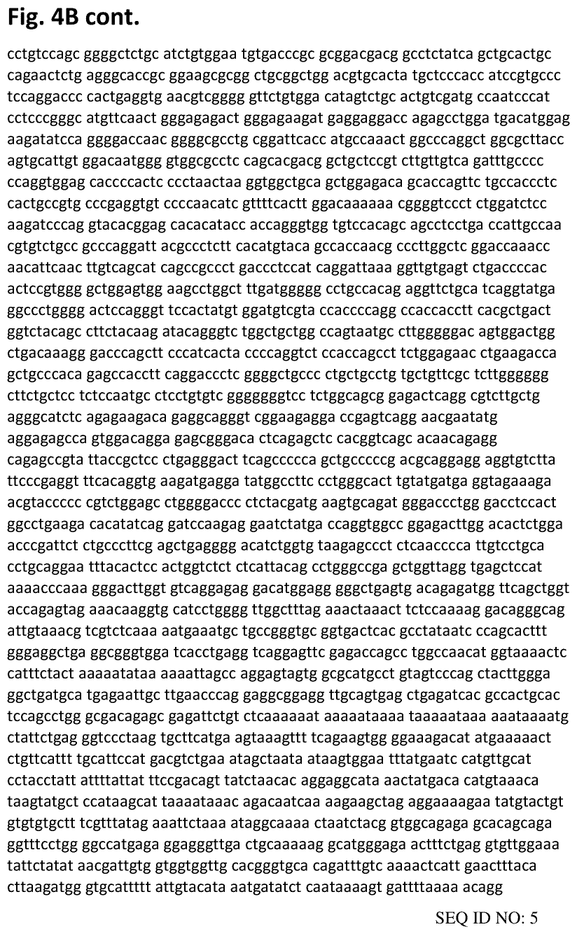

### EXAMPLES

A suitable AAV serotype will be used to transfect mouse and human podocytes in vitro, and mouse NS knockout models in vivo. The AAV plasmid will have a NPHS1 minimal promoter cassette, WPRE and bGH cassettes, and an SRNS CDNA sequence (i.e., a NS-associated transgene) cloned in.

### REFERENCES

M. KESTILÄ, U. LENKKERI, M. MÄNNIKKÖ, J. LAMERDIN, P. MCCREADY, H. PUTAALA, V. RUOTSALAINEN, T. MORITA, M. NISSINEN, R. HERVA, C.E. KASHTAN, L. PELTONEN, C. HOLMBERG, A. OLSEN, K. TRYGGVASON. 1998. Positionally cloned gene for a novel glomerular protein-nephrin-is mutated in congenital nephrotic syndrome. Mol Cell, 1 pp. 575-582
LUO, X., HALL, G., LI, S., BIRD, A., LAVIN, P. J., WINN, M. P., KEMPER, A. R., BROWN, T. T. & KOEBERL, D. D. 2011. Hepatorenal correction in murine glycogen storage disease type I with a double-stranded adeno-associated virus vector. Mol Ther, 19, 1961-70.
MOELLER, M. J., SANDEN, S. K., SOOFI, A., WIGGINS, R. C. & HOLZMAN, L. B. 2002. Two gene fragments that direct podocyte-specific expression in transgenic mice. J Am Soc Nephrol, 13, 1561-7.
PICCONI, J. L., MUFF-LUETT, M. A., WU, D., BUNCHMAN, E., SCHAEFER, F. & BROPHY, P. D. 2014. Kidney-specific expression of GFP by in-utero delivery of pseudotyped adeno-associated virus 9. Molecular Therapy Methods & Clinical Development, 1, 14014.
ROCCA, C. J., UR, S. N., HARRISON, F. & CHERQUI, S. 2014. rAAV9 combined with renal vein injection is optimal for kidney-targeted gene delivery: conclusion of a comparative study. Gene therapy, 21, 618-628.
SCHAMBACH, A., BOHNE, J., BAUM, C., HERMANN, F. G., EGERER, L., VON LAER, D. & GIROGLOU, T. 2005. Woodchuck hepatitis virus post-transcriptional regulatory element deleted from X protein and promoter sequences enhances retroviral vector titer and expression. Gene Therapy, 13, 641.
SCHIEVENBUSCH, S., STRACK, I., SCHEFFLER, M., NISCHT, R., COUTELLE, O., HÖSEL, M., HALLEK, M., FRIES, J. W. U., DIENES, H.-P., ODENTHAL, M. & BÜNING, H. 2010. Combined Paracrine and Endocrine AAV9 mediated Expression of Hepatocyte Growth Factor for the Treatment of Renal Fibrosis. Molecular Therapy, 18, 1302-1309.
VAN DER WOUDEN, E. A., SANDOVICI, M., HENNING, R. H., DE ZEEUW, D. & DEELMAN, L. E. 2004. Approaches and methods in gene therapy for kidney disease. J Pharmacol Toxicol Methods, 50, 13-24.

1-13. (canceled)

14. A method of treating a monogenic form of nephrotic syndrome comprising administering an adeno-associated virus (AAV) vector to a patient with the monogenic form of nephrotic syndrome by injection into a kidney artery of the patient, wherein the AAV vector targets podocytes within the glomerulus of the kidney and comprises:
a nephrotic syndrome-associated transgene; and
a minimal nephrin promoter or a minimal podocin promoter,
wherein the nephrotic syndrome-associated transgene is selected from one or more of NPHS1, TRPC6, NUP107, NUP133, NUP160, ACTN4, INF2, ANKFY1, ANLN, CRB2, ITGA3, KANK1, KANK4, MAGI2, MYO1E, OCRL, PTPRO, SMARCAL1, SYNPO, TBC1D8B, XPO5, TNS2 or NLRP3.

15. The method according to claim 14, wherein the AAV vector is AAV serotype 9, LK03 or 3B.

16. The method according to claim 14, wherein the AAV vector additionally comprises a Woodchuck hepatitis post-transcriptional regulatory element (WPRE).

17. The method according to claim 14, wherein the nephrotic syndrome-associated transgene is human.

18. The method according to claim 14, wherein the AAV vector additionally comprises a Kozak sequence between the promoter and the nephrotic syndrome-associated transgene.

19. The method according to claim 14, wherein the AAV vector additionally comprises a polyadenylation signal.

20. The method according to claim 14, wherein expression of the nephrotic syndrome-associated transgene is specifically targeted to podocytes within the glomerulus of the kidney and reverses the nephrotic syndrome phenotype and corrects podocyte-associated kidney dysfunction.

21. The method according to claim 14, wherein the monogenic form of nephrotic syndrome is a monogenic form of steroid-resistant nephrotic syndrome.

22. The method according to claim 14, wherein the patient is human.

23. The method according to claim 22, wherein the patient is a paediatric patient.

24. The method according to claim 22, wherein the patient is an adult patient.

25. An adeno-associated virus (AAV) vector comprising:
a nephrotic syndrome-associated transgene; and
a minimal podocin promoter,
wherein the nephrotic syndrome-associated transgene is selected from one or more of NPHS1, TRPC6, NUP107, NUP133, NUP160, ACTN4, INF2, ANKFY1, ANLN, CRB2, ITGA3, KANK1, KANK4, MAGI2, MYO1E, OCRL, PTPRO, SMARCAL1, SYNPO, TBC1D8B, XPO5, TNS2 or NLRP3.

26. The AAV vector according to claim 25, wherein the AAV vector is AAV serotype 9, LK03 or 3B.

27. The AAV vector according to claim 25, wherein the AAV vector additionally comprises a Woodchuck hepatitis post-transcriptional regulatory element (WPRE).

28. The AAV vector according to claim 25, wherein the nephrotic syndrome-associated transgene is human.

29. The AAV vector according to claim 25, wherein the AAV vector additionally comprises a Kozak sequence between the promoter and the nephrotic syndrome-associated transgene.

30. The AAV vector according to claim 25, wherein the AAV vector additionally comprises a polyadenylation signal.

31. A method of treating a monogenic form of nephrotic syndrome comprising administering an AAV vector according to claim 25 to a patient with the monogenic form of nephrotic syndrome.

32. The method according to claim 31, wherein expression of the nephrotic syndrome-associated transgene is specifically targeted to podocytes within the glomerulus of the kidney and reverses the nephrotic syndrome phenotype and corrects podocyte-associated kidney dysfunction.

33. The method according to claim 31, wherein the monogenic form of nephrotic syndrome is a monogenic form of steroid-resistant nephrotic syndrome.

34. The method according to claim 31, wherein the patient is human.

35. The method according to claim 34, wherein the patient is a paediatric patient.

36. The method according to claim 34, wherein the patient is an adult patient.

37. The method according to claim 31, wherein the AAV vector is administered systemically.

38. The method according to claim 31, wherein the AAV vector is administered by intravenous injection.

39. The method according to claim 31, wherein the AAV vector is administered by injection into the renal artery.
